(19) United States
(12) Patent Application Publication
(10) Pub. No.: US 2023/0309467 A1
(43) Pub. Date: Oct. 5, 2023

(54) FUNGI GROWTH CHAMBER

(71) Applicant: Michigan Myco, Southfield, MI (US)

(72) Inventors: Brandon Hannawa, Bloomfield Hills, MI (US); Matthew Hannawa, Bloomfield Hills, MI (US)

(21) Appl. No.: 17/889,432

(22) Filed: Aug. 17, 2022

Related U.S. Application Data
(60) Provisional application No. 63/325,850, filed on Mar. 31, 2022.

(51) Int. Cl. A01G 18/22; A01G 18/65; C12N 1/14

(52) U.S. Cl. CPC A01G 18/22; C12R 2001/645; C12N 1/14; A01G 18/65

(57) ABSTRACT

A fungal growth chamber includes a base, multiple walls extending upward from the base, and a cavity defined by the base and the walls. A lid contacts each of the walls and encloses the cavity. Multiple ventilation holes disposed about the walls. The plurality of ventilation holes includes a set of intake holes and a set of exhaust holes, and each hole in the set of exhaust holes is spaced farther from the base than each hole in the set of intake holes.

### CROSS-REFERENCE TO RELATED APPLICATION

This application claims priority to U.S. Provisional Patent Application No. 63/325,850 filed on Mar. 31, 2022.

### TECHNICAL FIELD

The present disclosure relates generally to growth chambers for fungi and/or micro-greens, and specifically to a growth chamber able to be used in a home mushroom growth kit.

### BACKGROUND

Plant propagation kits, including kits designed for growing fungi, in both small and medium growth operations typically rely on the environment in which the kit is placed to maintain temperature, air composition, and humidity requirements. Some plants or other growing organisms are more sensitive to environmental changes and contaminations than others, and the growth chambers and systems should be designed to accommodate the sensitivities. In kits designed for home use and/or use by someone who is not a horticulture expert, the sensitivity is exacerbated due to the inexperience of the intended user. Furthermore, currently available kits are often designed for propagation of larger plants and are tailored to the environmental needs of growing a larger plant. As a result, the kits are less suitable for fungi and microgreens.

Existing ventilated plant propagation assembly kits typically contain a tray with ribs as well as a dome with gaps for aeration in order to provide ideal plant growth sub-environments within a larger environment. The gaps are open air gaps in the domed lid and provide uninhibited and uncontrolled airflow. While this may be ideal for plants, it is not ideal for fungi, and similar growths.

The existing growth chambers are not designed to facilitate growth of fungi, and do not provide sufficient (or any) air filtration and moisture management. Nor do the existing systems include a way to measure environment parameters such as moisture and temperature. Further, the existing systems do not include a way to prevent the development of microclimates within the growth chamber.

### SUMMARY OF THE INVENTION

In one exemplary embodiment a fungal growth chamber includes a base, a plurality of walls extending upward from the base, a cavity defined by the base and the plurality of walls, a lid contacting each of the walls and enclosing the cavity, a plurality of ventilation holes disposed about the plurality of walls, wherein the plurality of ventilation holes includes a set of intake holes and a set of exhaust holes, and each hole in the set of exhaust holes being spaced farther from the base than each hole in the set of intake holes.

In another example of the above described fungal growth chamber the plurality of walls includes a set of end walls and a set of side walls, each of the end walls being connected to at least one other end wall via at least one side wall.

In another example of any of the above described fungal growth chambers each of the exhaust holes is disposed on an end wall.

In another example of any of the above described fungal growth chambers each of the intake holes is disposed on a side wall.

In another example of any of the above described fungal growth chambers each side wall includes a subset of the intake holes, and wherein the subset of intake holes are evenly spaced along the corresponding side wall.

In another example of any of the above described fungal growth chambers each intake hole is spaced apart from the base by at least 2.5 inches.

In another example of any of the above described fungal growth chambers each ventilation hole is covered by a filter.

In another example of any of the above described fungal growth chambers each filter extends beyond the circumference of the ventilation hole by at least 0.25 inches.

In another example of any of the above described fungal growth chambers each filter is disposed exterior to the cavity.

In another example of any of the above described fungal growth chambers each filter is comprised of a compressed polyester fibers and has an average pore size of 0.3 microns.

Another example of any of the above described fungal growth chambers further includes a non-permeable film covering each ventilation hole on a side of the corresponding wall, wherein the non-permeable film is removable.

In another example of any of the above described fungal growth chambers each of the ventilation holes is the same size as each other of the ventilation holes.

Another example of any of the above described fungal growth chambers further includes a liner disposed in the cavity, wherein the liner includes a lipped circumference.

In another example of any of the above described fungal growth chambers each end wall includes a subset of the exhaust holes, and wherein the subset of exhaust holes are evenly spaced along the corresponding end wall.

In another example of any of the above described fungal growth chambers each exhaust hole is spaced apart from the base by at least 3.4 inches.

In another example of any of the above described fungal growth chambers each end wall has a shorter dimension along a circumference of the base than each side wall.

In another example of any of the above described fungal growth chambers the lid is configured to contact each of the walls and enclose the cavity in a first sealed configuration and a second unsealed configuration.

In another example of any of the above described fungal growth chambers a gasket is disposed about the circumference of the lid, and the gasket seals the lid against each of the walls, in the first configuration.

In another example of any of the above described fungal growth chambers the contact between the lid and each of the walls defines at least one gap in the second configuration such that air exchange between an exterior environment and the interior of the cavity occurs while the lid is in the second configuration.

Another example of any of the above described fungal growth chambers further includes a hygrometer and thermometer probe disposed in the lid, the hygrometer and thermometer probe including a screen visible from an exterior of the fungal growth chamber.

### BRIEF DESCRIPTION OF THE DRAWINGS

FIG. 1 schematically illustrates an isometric view of an example growth chamber for a fungi growth kit.

FIG. 2 schematically illustrates an end of the example growth chamber of FIG. 1.

FIG. 3 schematically illustrates a side of the example growth chamber of FIG. 1.

FIG. 4 schematically illustrates an exhaust hole positioned on the end wall of FIG. 2.

FIG. 5 schematically illustrates an intake hole positioned on the side illustrated in FIG. 3.

FIG. 6 schematically illustrates an exemplary lid for the growth chamber of FIG. 1.

FIG. 7 schematically illustrates airflow through the growth chamber of FIG. 1.

The embodiments, examples and alternatives of the preceding paragraphs, the claims, or the following description and drawings, including any of their various aspects or respective individual features, may be taken independently or in any combination. Features described in connection with one embodiment are applicable to all embodiments, unless such features are incompatible. Like reference numbers and designations in the various drawings indicate like elements.

### DETAILED DESCRIPTION

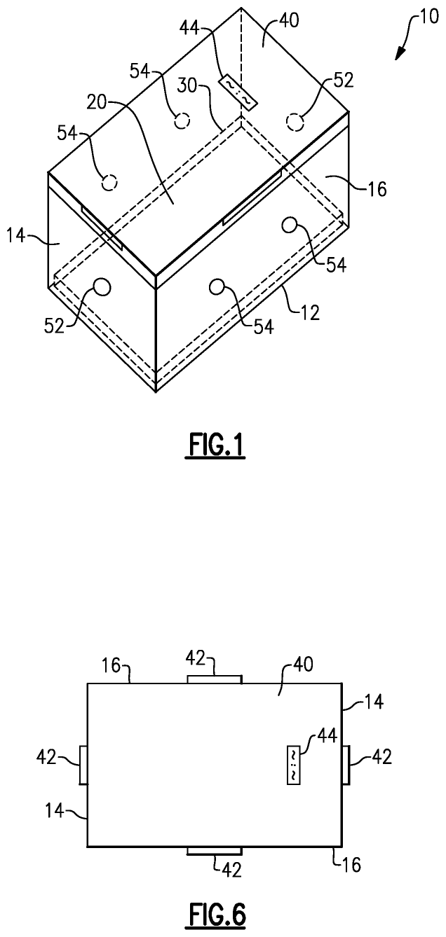

FIG. 1 schematically illustrates an exemplary fungi growth chamber 10 including a cavity 20. The cavity 20 provides a controlled chamber for growing fungi. The growth chamber 10 expels CO₂ and intakes O₂ using a passive process known as fresh air exchange (FAE). The passive process is controlled by the physical construction of the chamber 10 and placement of intake and exhaust ventilation holes. In addition, the chamber 10 measures relative humidity and temperature of the growth chamber and provides the measured information to a user outside of the chamber via a screen on a thermometer probe 44.

The growth chamber 10 includes a base 12. Multiple walls 14, 16 extend upwards from the base 12. The walls 14, 16 include a set of side walls 16 and a set of end walls 14. In the illustrated examples, the side walls 16 are longer than the end walls 14 and the chamber 10 is rectangular. In other examples, alternate chamber 10 shapes with alternate counts of walls can be utilized with the end walls 14 being defined by the presence of exhaust holes 52, and the side walls 16 being defined by the presence of intake holes 54, and each end wall 14 being connected to two side walls.

The walls 14, 16 and the base 12 define a cavity 20. Positioned within the cavity 20 is a liner 30. The liner 30 provides a receptacle for receiving fungal growth components and separating the growth components from the base 12 in order to prevent contamination of the growth components and to assist in maintaining proper conditions for growth.

To further maintain a desirable environment that facilitates growth of a fungi and microgreens by preventing the formation of microclimates and reducing risk of contamination, the chamber 10 is enclosed by a lid 40. The lid 40 includes a hygrometer and thermometer probe 44 that monitors the interior temperature and moisture levels within the cavity 20. The probe 44 displays the monitored temperature and moisture levels such that a user can determine the temperature and moisture levels within the cavity 20 without opening the lid 40, and thereby without accidentally altering the internal environment of the cavity 20.

In some examples, the lid 40 can include multiple configurations by which the lid encloses the chamber 10. In a first configuration, a first side of the lid 40 is facing outwards and the circumference of the lid 40 is sealed to each of the walls 14,16. The seal prevents air exchange into and out of the chamber 10 at the circumference of the lid 40. In one example, the seal includes a gasket around the circumference of the lid 40.

At some steps of the growing process it can be desirable to allow some air exchange at the edges of the lid 40. In order to facilitate this exchange, the lid is reversible and can enclose the chamber in a second configuration with a second side facing outwards. In the second configuration, the circumference of the lid 40 includes at least one gap that allows air exchange to occur between the lid 40 and the walls 14, 16.

In examples using the lid 40 having multiple configurations, the hygrometer and thermometer probe 44 includes a screen on one side and can be removed and replaced from either side of the lid 40 allowing the user to view the measured information regardless of which configuration the lid 40 is in.

Also disposed on the end walls 14 and the side walls 16, are a set of ventilation holes including exhaust holes 52 and intake holes 54. The exhaust holes 52 and intake holes 54 are spaced about the walls 14, 16 in a manner to direct airflow through the cavity 20. The airflow is directed by the relative placement of the exhaust holes 52 and intake holes 54. Warm carbon dioxide expelled by the growing fungi within the cavity 20 weighs less than the fresh oxygen required by the growing fungi. By positioning the exhaust holes 52 on the end walls 14 higher than the intake holes 54 on the side walls 16, relative to the bottom of the growth chamber 10, a passive airflow pattern (illustrated in FIG. 7) is generated. The passive airflow pattern allows for continuous circulation of cool, oxygen rich air into the cavity 20 and exhaustion of warm, carbon dioxide rich air out of the chamber along the illustrated flow lines 22.

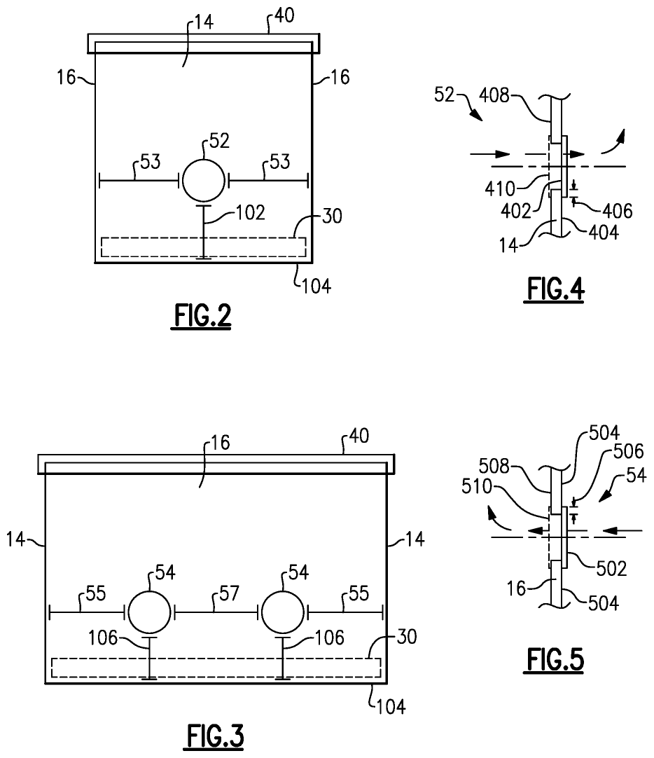

With continued reference to FIGS. 1 and 7, and with like numerals indicating like elements, FIG. 2 illustrates an end wall 14 demonstrating placement of the exhaust hole 52. Similarly, FIG. 3 illustrates a side wall 16 demonstrating a placement of the intake holes 54. Each exhaust hole 52 is placed an equal distance 53 from each side wall 16, and is a first height 102 from the base 104. In one example, the first height is 3.4 inches from the base to a bottom edge of the hole. Each intake hole 54 is placed a second distance 55 from the end walls 14, and third distance from the other of the intake holes 54. In alternative examples using alternative numbers of intake holes 54 and/or exhaust holes 52, the distribution of the ventilation holes 52, 54 can be evenly dispersed about the circumference of the walls 14, 16 in order to provide the proper passive airflows to encourage healthy growth within the cavity 20. In some examples desirable airflows are achieved in part by making each ventilation hole the same size. In one specific example, the desirable airflows are achieved by making each ventilation hole 52, 54 1.5 inches in diameter. Each of the intake holes 54 is placed a fourth height 106 upward from the base 104. The fourth height 106 is less than the first height 102, allowing for the heavier oxygen rich air entering the cavity 20 to push the lighter carbon dioxide rich air exiting the cavity 20 out the exhaust holes 52 along the airflow lines 22. In one example, the fourth height 106 is 2.5 inches from the base to an edge of the intake hole 54.

The fourth height 106 positions the intake holes 54 above the top of the liner 30 in order to further facilitate the desirable passive airflows. In the illustrated example of FIGS. 2 and 3, the intake holes 54 are positioned entirely above the top of the liner 30. In alternate, examples, the intake holes 54 can be disposed such that they are only partially above the top of the liner 30 and achieve similar passive airflow benefits.

With continued reference to FIGS. 1-3, FIG. 4 illustrates an exemplary exhaust hole 52, including a filter 402 disposed on an exterior surface 404 of the wall 14. The filter 402 overlaps with the wall 14 by an overlap 406 of at least 0.25 inches along the circumference of the filter 402, and is secured in position using an adhesive. In one example, the filter 402 is constructed of compressed polyester fibers having a 0.3 micron pore size. In alternative examples, having different airflow requirements, alternative materials and/or pore sizes can be utilized for the filter. The filter 402 minimizes the entry of any contaminants while still allowing for relatively free flow of air through the cavity 20. In alternative examples, the filter 402 can be placed on the interior surface 408 of the wall 14 and function similarly.

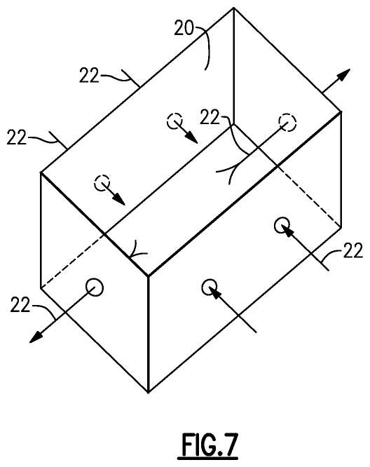

With continued reference to FIGS. 1-4, FIG. 5 illustrates an exemplary filter configuration for an intake hole 54. In the example of FIG. 5, a filter 502 is disposed on an exterior surface 504 of the side wall 16. As with the exhaust hole 52 of FIG. 4, the filter overlaps the exterior wall via an overlap distance 506 of at least 0.25 inches, and an adhesive secures the filter 502 to the exterior surface 504 of the wall 16. As with the exhaust filter 402, the filter 502 is constructed of compressed polyester fibers having a 0.3 micron pore size. In alternative examples, having different airflow requirements, alternative materials and/or pore sizes can be utilized for the filter. The filter 502 minimizes the entry of contaminants into the cavity 20 though the intake hole 54.

During an initial growing phase, it can be beneficial to prevent air from entering into or exhausting from the cavity 20. Prevention of air exchange during the early growing phases ensures that the growth components remain uncontaminated. In such an example, a removable barrier 410, 510 is disposed on the interior surface 408, 508 of the walls 14, 16. The barrier 410, 510 prevents airflow through the hole 52, 54 and is constructed of an impermeable material such as plastic. The adhesive connecting the barrier 410, 510 to the interior surface 508 of the wall 14, 16 is a weak adhesive allowing an individual to remove the barrier 410, 510 manually after the initial growing phases have completed. In alternative examples, the barrier 410, 510 itself can be structurally weak, while the adhesive is strong. In this example, the barrier 410, 510 is removed by destruction of the barrier 410, 510. In yet further alternate examples, the positions of the barrier 410, 510 and the corresponding filter 402, 502 can be exchanged, allowing for the barrier 410, 510 to be removed without opening the lid 40.

With continued reference to FIGS. 1-5, and with like numerals indicating like elements, FIG. 6 illustrates an exemplary lid 40. In order to ensure a controlled environment with correctly targeted passive airflows within the cavity 20, the lid 40 in the illustrated example includes manual seals 42 on each wall 14, 16. The manual seals 42 can be engaged by the user to prevent access and airflow into the top of the cavity 20, or disengaged to allow access to the cavity 20. The access is used to add components or adjust components during various stages of the growing process.

Also included on the lid 40 is a hygrometer and thermometer probe 44. The hygrometer and thermometer probe 44 includes a set of sensors that measure the temperature and moisture content within the cavity 20, and a screen that displays the measured values. The screen is visible without opening the cavity 20 in order to allow the conditions to be monitored without altering them.

With reference to the full structure described above, and illustrated in FIGS. 1-7, the precisely placed ventilation holes on the chamber walls create a positive pressure system to expel CO₂ and intake O₂. Synthetic filter patches control airflow into and out of the chamber and prevent bacteria and other contaminants from entering the chamber. The filters further maintain good growth conditions in the chamber by assisting in maintaining moisture and temperature levels. A built-in and reversible hygrometer-thermometer measures the relative humidity and temperature within the growth chamber and displays those measurements to the user. A plastic liner at the base of the chamber can shrink with the medium to prevent side pins, microclimates, and contaminants from forming. The liner further provides for easy cleanup after the growth, which enhances the reusability of the chamber.

The primary components of one example embodiment include: Multiple 1.5″ airflow holes, with two or more airflow holes on each long side and one airflow hole on each short side. The airflow holes on the long sides are 2.5″ up from a base of the chamber. The airflow holes on the short sides are 3.4″ up from the base of the chamber. In a more generic example, the airflow holes on the short side are placed higher up than the airflow hole on the long side to facilitate proper directionality of airflow due to the pressure differential. The long side holes are evenly distributed along each long side. The short side holes are centered on the short sides. Placed over each airflow hole is a corresponding 2.0″ synthetic filter patch. The synthetic filter patches filter the airflow into and out of the holes and prevent contaminants from entering the growth chamber during the growing process. Included within the growth chamber is a plastic liner that is positioned on the base of the growth chamber and configured to receive and retain growth components. The lid on the growth chamber includes an integrated and reversible hygrometer-thermometer configured to determine and display a moisture content and a temperature of the interior of the growth chamber. The display is visible to a person exterior to the chamber without requiring the person to open the chamber.

It is further understood that any of the above described concepts can be used alone or in combination with any or all of the other above described concepts. Although an embodiment of this invention has been disclosed, a worker of ordinary skill in this art would recognize that certain modifications would come within the scope of this invention. For that reason, the following claims should be studied to determine the true scope and content of this invention.

1. A fungal growth chamber comprising:
a base;
a plurality of walls extending upward from the base;
a cavity defined by the base and the plurality of walls;
a lid contacting each of the walls and enclosing the cavity;
a plurality of ventilation holes disposed about the plurality of walls, wherein the plurality of ventilation holes includes a set of intake holes and a set of exhaust holes; and
each hole in the set of exhaust holes being spaced farther from the base than each hole in the set of intake holes.

2. The growth chamber of claim 1, wherein the plurality of walls includes a set of end walls and a set of side walls, each of the end walls being connected to at least one other end wall via at least one side wall.

3. The fungal growth chamber of claim 2, wherein each of the exhaust holes is disposed on an end wall.

4. The fungal growth chamber of claim 3, wherein each of the intake holes is disposed on a side wall.

5. The fungal growth chamber of claim 1, wherein each side wall includes a subset of the intake holes, and wherein the subset of intake holes are evenly spaced along the corresponding side wall.

6. The fungal growth chamber of claim 5, wherein each intake hole is spaced apart from the base by at least 2.5 inches.

7. The fungal growth chamber of claim 1, wherein each ventilation hole is covered by a filter.

8. The fungal growth chamber of claim 7, wherein each filter extends beyond the circumference of the ventilation hole by at least 0.25 inches.

9. The fungal growth chamber of claim 7, wherein each filter is disposed exterior to the cavity.

10. The fungal growth chamber of claim 7, wherein each filter is comprised of a compressed polyester fibers and has an average pore size of 0.3 microns.

11. The fungal growth chamber of claim 7, further comprising a non-permeable film covering each ventilation hole on a side of the corresponding wall, wherein the non-permeable film is removable.

12. The fungal growth chamber of claim 1, wherein each of the ventilation holes is the same size as each other of the ventilation holes.

13. The fungal growth chamber of claim 1, further comprising a liner disposed in the cavity, wherein the liner includes a lipped circumference.

14. The fungal growth chamber of claim 1, wherein each end wall includes a subset of the exhaust holes, and wherein the subset of exhaust holes are evenly spaced along the corresponding end wall.

15. The fungal growth chamber of claim 14, wherein each exhaust hole is spaced apart from the base by at least 3.4 inches.

16. The fungal growth chamber of claim 1, wherein each end wall has a shorter dimension along a circumference of the base than each side wall.

17. The fungal growth chamber of claim 1, wherein the lid is configured to contact each of the walls and enclose the cavity in a first sealed configuration and a second unsealed configuration.

18. The fungal growth chamber of claim 17, wherein a gasket is disposed about the circumference of the lid, and the gasket seals the lid against each of the walls, in the first configuration.

19. The fungal growth chamber of claim 17, wherein the contact between the lid and each of the walls defines at least one gap in the second configuration such that air exchange between an exterior environment and the interior of the cavity occurs while the lid is in the second configuration.

20. The fungal growth chamber of claim 1, further comprising a hygrometer and thermometer probe disposed in the lid, the hygrometer and thermometer probe including a screen visible from an exterior of the fungal growth chamber.
